(19) United States
(12) Patent Application Publication
(10) Pub. No.: US 2013/0012694 A1
(43) Pub. Date: Jan. 10, 2013

(54) MONUMENTAL ADORNMENT

(75) Inventor: Fang Liang ZHANG, Fanwood, NJ (US)

(73) Assignee: NANJINGJINSIRUI SCIENCE & TECHNOLOGY BIOLOGY CORP., Nanjing (CN)

(21) Appl. No.: 13/542,289

(22) Filed: Jul. 5, 2012

Related U.S. Application Data
(60) Provisional application No. 61/504,353, filed on Jul. 5, 2011.

(30) Foreign Application Priority Data
Sep. 26, 2011 (CN) 201110288949.2
Sep. 26, 2011 (CN) 201120363964.4

(51) Int. Cl. C07H 21/04; C07H 1/08

(52) U.S. Cl. 536/23.5; 536/25.3

(57) ABSTRACT

Monumental adornments carrying an isolated genetic material of an organism, such as a human being, a non-human animal, a plant or a microorganism are described. Also described are methods of producing the monumental adornments.

### CROSS-REFERENCE TO RELATED APPLICATIONS

This application claims the benefit under 35 U.S.C. §119(e) of U.S. Provisional Application No. 61/504,353, filed Jul. 5, 2011, and the benefit under 35 U.S.C. §119(a) of Chinese Patent Application Numbers 201120363964.4 and 201110288949.2, both filed Sep. 26, 2011, the entire disclosure of all of these applications is hereby incorporated herein by reference.

### BACKGROUND OF THE INVENTION

This invention relates generally to the field of adornments, and more specifically to adornments carrying isolated genetic materials, such as DNA molecules isolated from human beings, non-human animals or plants.

Life is full of special moments. Some persons or things deserve more than a memory. Whether celebrating a win, marking a special event or paying tribute to a precious personal bond, one may want to capture something forever. Thus, having a memento that contains a part of the special person, animal, plant, etc., would be of great value.

United States patent application 2008/0282733 describes a memory gemstone having carbon-containing gas produced from human or animal tissues as well as a method of producing the memory gemstone.

Deoxyribonucleic acid, or DNA, is a nucleic acid that contains the genetic instructions used in the development and functioning of almost all known living organisms on earth (with the exception of RNA viruses and prion). DNA is thus recognized as the universal genetic code that differentiates one from the others, representing the ultimate individuality of each person. The main role of DNA molecules is the long-term storage of information. DNA is often compared to a set of blueprints, like a recipe or a code, because it contains the instructions needed to construct other components of cells, such as proteins and RNA molecules. DNA segments that carry the genetic information encoding a protein or RNA are called genes. DNA also contains certain sequences whose function is to be recognized by regulator molecules, such as proteins or RNAs. Both gene sequences and sequences recognized by regulator molecules constitute genetic information.

It would be of great commemorative significance to place an isolated genetic material, such as the DNA, RNA or prion, of a special person, a special pet or any other special organism in a monumental adornment, such as a piece of jewelry, decorative artifacts or other arts and crafts. Such a monumental adornment creates an everlasting connection and helps to commemorate that special person, animal or other organism. In addition, the adornment provides a means to preserve the genetic material for other purposes, such as identification or amplification, in the future.

### BRIEF SUMMARY OF THE INVENTION

One general aspect of the present invention relates to a monumental adornment comprising an isolated genetic material of an organism.

Another general aspect of the present invention relates to a method of preparing a monumental adornment, comprising:

a. isolating a genetic material from an organism; and

b. combining the isolated genetic material with at least one material to produce the monumental adornment.

The monumental adornment according to embodiments of the present invention, can comprise an isolated DNA molecule of a human being, a non-human animal, a plant or a microorganism.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 illustrates a longitudinal cross-section diagram of a ring (1) according to an embodiment of the present invention, which has an isolated genetic material (3) linked directly to a surface area (2) of the ring;

FIG. 2 illustrates a stereogram of the ring shown in FIG. 1;

FIG. 3 illustrates a longitudinal cross-section diagram of a ring (1) according to another embodiment of the present invention, where an isolated genetic material (3) is placed on a carrier (4), which is fixed to a surface area of the ring via a lock and support mechanism (2);

FIG. 4 is a longitudinal cross-section diagram of a pendant (1) according to an embodiment of the present invention, where an isolated genetic material (3) is linked directly to a surface area of a compartment (5) within the adornment;

FIG. 5 is a horizontal cross-section diagram of the pendant (1) shown in FIG. 4;

FIG. 6 is a longitudinal cross-section diagram of a pendant (1) according to another embodiment of the present invention, where an isolated genetic material (3) is placed on a carrier (4), which is fixed to a surface area of a compartment (5) within the adornment via a lock and support mechanism (2);

FIG. 7 is a horizontal cross-section diagram of the pendant (1) shown in FIG. 6;

FIG. 8 is picture of a gel containing the genomic DNA (2^{nd} and 3^{rd} lanes) isolated from the mouse blood according to an embodiment of the present invention, the 1^{st} lane contains DNA markers; and

FIG. 9 is picture of a gel containing the genomic DNA (2^{nd} and 3^{rd} lanes) isolated from the mouse liver according to an embodiment of the present invention, the 1^{st} lane contains DNA markers.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

A general aspect of the present invention relates to a monumental adornment comprising an isolated genetic material of an organism.

As used herein, an “isolated genetic material” refers to a genetic material, such as a nucleic acid molecule or a polypeptide, that is substantially separated from at least one of the other materials present in the natural source of the genetic material, or is substantially free of at least one of the chemical precursors or other chemicals when the genetic material is chemically synthesized. A genetic material is “substantially separated from” or “substantially free of” another material or another chemical when there is less than about 30%, 20%, 10%, or 5% or less, by dry weight, of the other material or the other chemical. The genetic material plays a fundamental role in determining the structure and nature of a cell of an organism, and is capable of self-propagating and variation. Examples of isolated genetic materials include isolated DNA molecules, RNA molecules and prion proteins. In a preferred embodiment, the isolated genetic material is DNA. The isolated DNA can be in a single-strand or double-stranded form. It can be the whole genomic DNA or fragments of genomic DNA.

The genetic material of any organism of interest can be carried by a monumental adornment according to embodiments of the present invention. In one embodiment, the genetic material is isolated from one or more human beings, such as a loved one. In another embodiment, the genetic material is isolated from one or more non-human animals, such as a pet. In yet another embodiment, the genetic material is isolated from one or more organisms involved a memorable event, such as the whales or organisms died in a catastrophic event, etc.

The non-human animals include, but are not limited to, Mammalia, Ayes, Reptilia, Amphibia, Gnathostomata, Agnatha, Cephalochorda, Thaliacea, Ascidiacea, Appendiculariae, Graptolite, Pterobranchia, Enteropneusta, Asteroidea, Ophiuroidea, Echinoidea, Holothuroidea, Blastoidea, Crinoidea, Archisagittoidea, Sagittoidea, Hyolitha, Tentaculita, Rostroconchia, Scaphopoda, Cephalopoda, Bivalvia, Gastropoda, Aplacophora, Monoplacophora, Polyplacophora, Sipunculidea, Phascolosomatidea, Hirudinea, Oligochaeta, Polychaeta, Paleonemertea, Anopla, Monogonta, Digononta, Gymnolaemata, Phylactolaemata, Articulata, Inarticulata, Phasmida, Aphasmida, Eutardigrada, Mesotardigrada, Heterotardigrada, Malacostraca, Brabchiura, Mystacocarida, Copepoda, Ostracoda, Cirripedia, Branchiopoda, Cephalocarida, Protura, Diplura, Collembola, Insecta, Symphyla, Pauropoda, Diplopoda, Chilopoda, Pycnogonida, Merostomata, Arachnida, Trilobita, Cestoda, Trematoda, Turbellaria, Anthozoa, Tentaculata, Nuda, Scyphozoa, Hydrozoa, Demospongiae, Hexactinellida and Calcarer.

Preferably, the non-human animal is selected from the group consisting of monkey, dog, cat, rabbit, rat, mouse, squirrel, fish, snake, lizard, gecko, bird, pig, tortoise, horse, sheep, insect, procyon lotor, and koala.

In another embodiment, the genetic material is isolated from one or more plants, such as a follower plant or tree.

In yet another embodiment, the genetic material is isolated from one or more microorganisms.

According to an embodiment of the present invention, the monumental adornment comprises an isolated genetic material from a single organism.

In another embodiment of the present invention, the monumental adornment comprises isolated genetic materials from more than one organisms. Preferably, the more than one organisms have a special or significant connection, such as a couple, family members, etc.

The monumental adornment according to embodiments of the present invention can be any type of accessory or ornament. such as jewelries (necklace, ear rings, rings, pins, etc.), decorative artifacts (for rooms, cars, etc.), or other arts and crafts. The monumental adornment can be made of one or more suitable materials, such as a gem, agate, amber, crystal, metal, pearl, wood, bone, shell, leather, ceramics, glass, resin, rubber, paper, plastics, textiles and fiber. These material can be natural products or synthetic.

A gemstone or gem is a mineral, rock, or petrified material that when faceted, e.g., cut, and polished, is a collectible or can be used in jewelry. Gemstones can be used in the present invention include, but are not limited to, diamonds, rubies, emeralds, sapphires, amethyst, opals, aquamarine, tanzanite, and alexandrite. Gems are classified into different groups, species, and varieties. Some gemstones are manufactured or synthesized.

Metals that can be used in a monumental adornment include, but are not limited to, platinum, gold, silver, copper, aluminum, titanium, iron, or an alloy thereof.

The monumental adornment according to an embodiment of the present invention can be used for various purposes, such as creating a connection with an organism, commemorating the organism, or providing a means to preserve the genetic material for other purposes, such as identification or amplification of the organism, in the future. When needed, the genetic material can be isolated and amplified from the monumental adornment to obtain genetic information about the organism.

Another aspect of the present invention relates to a method of producing a monumental adornment. The method comprises isolating a genetic material from an organism; and combining the isolated genetic material with one or more materials to produce the monumental adornment.

The genetic material can be isolated from an organism of interest using any method known in the art, in view of the present disclosure. Standard molecular biology technology can be used in isolating the genetic material. From example, the blood/cell/tissue genomic DNA extraction kit from Tian-Geng Bio-tech (Beijing) Limited can be used to extract DNA from a whole blood sample or an oral epithelial cell sample of a mammal.

In certain embodiments, the isolated genetic material is unprocessed, and is spotted directly on a surface (exterior or interior) of an adornment via one or more active binding groups, e.g., by using the same technology as that in the manufacture of gene chip.

In other embodiments, the isolated genetic material is processed to increase its stability or is freeze-dried prior to being placed in a monumental adornment. Processed or freeze-dried isolated genetic material is then spotted or fixed to the adornment.

According to an embodiment of the present invention, the isolated genetic material, such as an isolated DNA molecule, processed or unprocessed, can be first associated with a nano-particle, which is then linked to, coated on, or embedded within the adornment. Materials that can be used for the preparation of nano-particles include, but are not limited to a metallic substance, such as gold, silver and bronze; a polymer, such as methyl methacrylate, poly lactic acid, etc; a resin formed with an inorganic salt; magnetic substance, etc. Embedding an isolated genetic material, such as a DNA, within a nano-particle, can keep the genetic material for longer periods of time without degradation, e.g., holding the isolated genetic material in the adornment for more than decades.

According to another embodiment of the present invention, to facilitate the production of the adornment and/or future analysis of the genetic material carried by adornment, the isolated genetic material, such as an isolated DNA molecule, processed or unprocessed, is first associated with a carrier. The carrier can be made of any suitable materials, such as paper, cloth, plastics, resin, rubber, wax, gem, etc., with any desirable shape and design, such as a box, a component of a support and lock mechanism, a platform, etc. The carrier with the isolated genetic material is then fixed to or placed in the adornment via any suitable mechanism, such as a glue, support and lock mechanism, etc. The carrier can be fixed to or placed at various locations of the adornment, such as the exterior or interior surface or a compartment of the adornment.

In certain embodiments, the parts of the adornment that are free of the isolated genetic material can be purchased or prepared separately from the preparation of the isolated genetic material. This will speed up and simply the preparation of the adornment, and make the process more acceptable to the general public.

In other embodiments, the isolated genetic material, such as a DNA molecule, is mixed with one or more materials that make up a portion or whole of the adornment.

An isolated genetic material can be combined with or placed into a monumental adornment or a carrier via various means in view of the present disclosure. For example, the isolated genetic material, such as an isolated DNA molecule, can be linked to or coated on the surface of an existing adornment by direct attachment or other chemical means. It can also be embedded in an existing adornment having an interior surface or a compartment. For example, a DNA molecule can be linked to an external or interior surface of the adornment directly, or indirectly via one or more active DNA-binding groups. Examples of the active groups include, but are not limited to one or more selected from the groups consisting of —COOH group, —C—O—C— group, —CHO group, and —OH group.

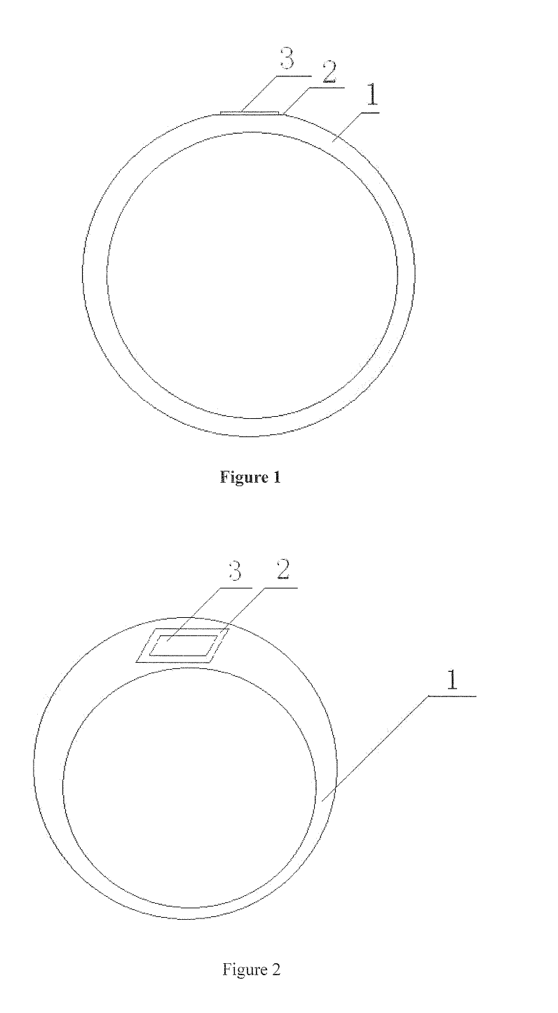

Referring to FIGS. 1 and 2, according to an embodiment of the present invention, an isolated genetic material (3), which can be a DNA, RNA or prion depending on the property of the organism of interest, is linked directly to an exterior surface area (2) of a ring (1). The isolated genetic material can also be linked to more than one surface areas of the ring at any location, such as at the center or periphery of the exterior or interior surface of the ring. The one or more surface areas can have or form a desired design. The isolated genetic material can also be linked to or coated over the entire exterior and/or interior surface of the ring.

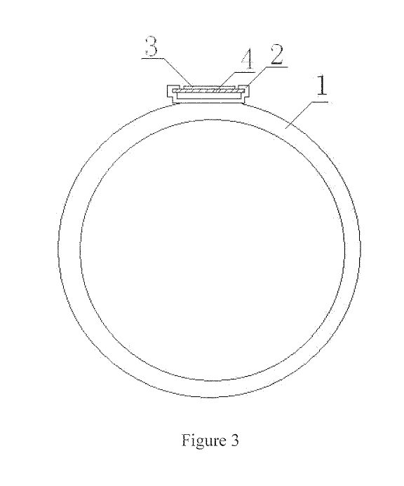

Referring to FIG. 3, according to another embodiment of the present invention, an isolated genetic material (3) is first placed on a carrier (4), which is then fixed to an exterior surface area of a ring (1) via a support and lock mechanism (2). The isolated genetic material can be placed on more than one surface areas of the ring via multiple support and lock mechanisms, each of which can fix one or more carriers, and each of the carriers can carry one or more types of genetic materials isolated from one or more organisms of interest.

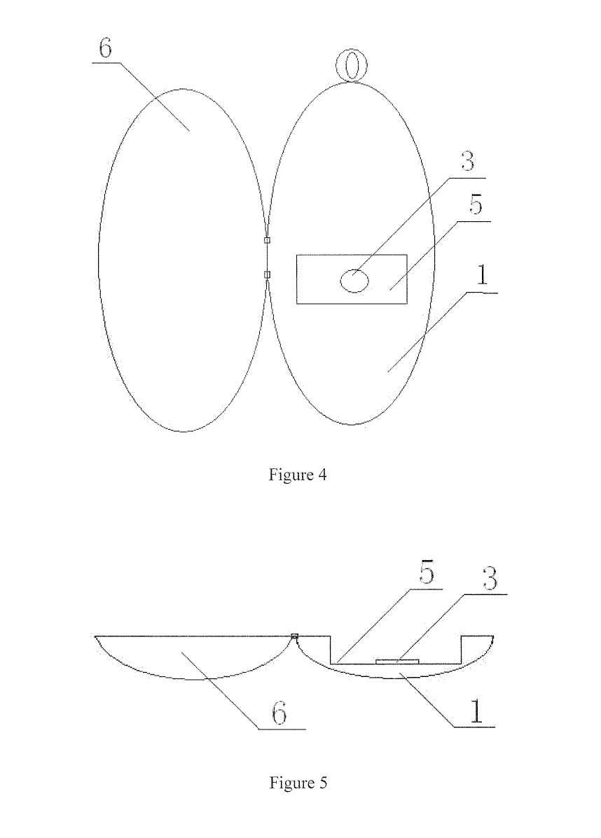

Referring to FIGS. 4 and 5, according to another embodiment of the present invention, an isolated genetic material (3) is linked directly to a surface area of a compartment (5) within a pendent (1). The isolated genetic material can be placed over a portion or the entire interior surface of the pendent. It can be linked to more than one surface areas within one or more compartments within the pendant. The pendent can be openable or sealed. The lid (6) or the side (6) opposing to the genetic material containing surface can be clear, opaque or solid.

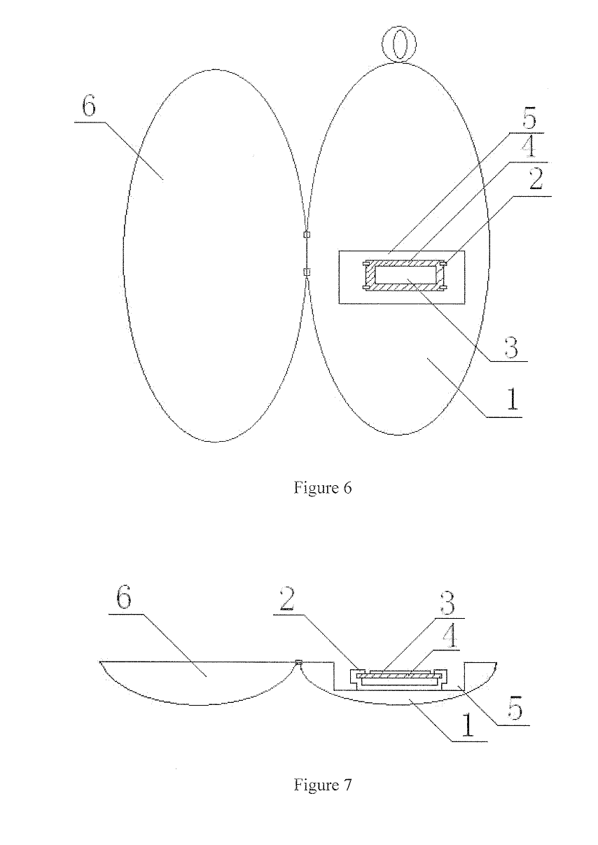

Referring to FIGS. 6 and 7, according to another embodiment of the present invention, an isolated genetic material (3) is first placed on a carrier (4), which is then fixed to a surface area of a compartment (5) within a pendent (1) via a support and lock mechanism (2). There can be more than one compartments for the placement of the isolated genetic material. The isolated genetic material can be placed on more than one surface areas of the compartment via multiple support and lock mechanisms, each of which can fix one or more carriers, and each of the carriers can carry one or more types of genetic materials isolated from one or more organisms of interest.

If the isolated genetic material is to be used for future identification purpose, the isolated genetic material is preferably placed in a compartment of the adornment, e.g., directly by linking it to a surface area of the compartment, or indirectly by placing a carrier containing the isolated genetic material in the compartment. The compartment can be openable or sealed.

According to another embodiment of the present invention, the isolated genetic material, such as DNA, is mixed with one or more materials that make up a portion or the entirety of an adornment. The isolated genetic material is therefore embedded in the portion or the entirety of the adornment.

Various embodiments of the invention have now been described. It is to be noted, however, that this description of these specific embodiments is merely illustrative of the principles underlying the inventive concept. It is therefore contemplated that various modifications of the disclosed embodiments will, without departing from the spirit and scope of the invention, be apparent to persons skilled in the art.

The following specific examples of the invention are further illustrative of the nature of the invention, it needs to be understood that the invention is not limited thereto.

### EXAMPLE 1

### Extraction of Genomic DNA from the Whole Blood

1. Blood samples are typically obtained as 1 ml of whole blood stored in EDTA vacutainer tubes frozen at −70° C.

2. Thaw the frozen samples, and to each 1 ml sample, add 0.8 ml 1×SSC buffer, and mix. Centrifuge for 1 minute at 12,000 rpm in a microcentrifuge.

3. Remove 1 ml of the supernatant and discard into disinfectant.

4. Add 1 ml of 1×SSC buffer, vortex, centrifuge as above for 1 minute, and remove all of the supernatant.

5. Add 375 ul of 0.2M NaOAc to each pellet and vortex briefly. Then add 25 ul of 10% SDS and 5 ul of proteinase K (20 mg/ml H2O) (Sigma P-0390), vortex briefly and incubate for 1 hour at 55 degC.

6. Add 120 ul phenol/chloroform/isoamyl alcohol and vortex for 30 seconds. Centrifuge the sample for 2 minutes at 12,000 rpm in a microcentrifuge tube.

7. Carefully remove the aqueous layer to a new 1.5 ml microcentrifuge tube, add 1 ml of cold 100% ethanol, mix, and incubate for 15 minutes at −20 deg C.

8. Centrifuge for 2 minutes at 12,000 rpm in a microcentrifuge. Decant the supernatant and drain.

9. Add 180 ul 10:1 TE buffer, vortex, and incubate at 55 degC. for 10 minutes.

10. Add 20 ul 2 M sodium acetate and mix. Add 500 ul of cold 100% ethanol, mix, and centrifuge for 1 minute at 12,000 rpm in a microcentrifuge.

11. Decant the supernatant and rinse the pellet with 1 ml of 80% ethanol. Centrifuge for 1 minute at 12,000 rpm in a microcentrifuge.

12. Decant the supernatant, and dry the pellet in a Speedy-Vac for 10 minutes (or until dry).

13. Resuspend the pellet by adding 200 ul of 10:1 TE buffer. Incubate overnight at 55 degC., vortexing periodically to dissolve the genomic DNA.

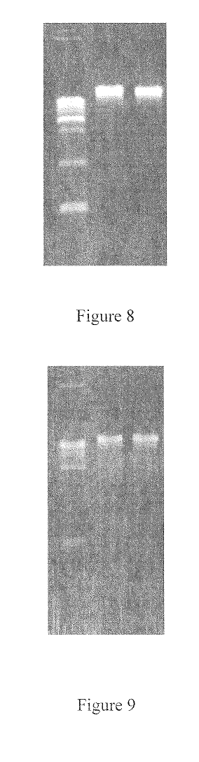

The isolated DNA is subject to electrophoresis analysis. FIG. 8 is picture of a gel containing the genomic DNA (2^{nd} and 3^{rd} lanes) isolated from a sample of mouse blood.

### EXAMPLE 2

### Extraction of Genomic DNA from Fresh Tissues

1. Weigh 0.5-1 g fresh tissue and put in motar. Add liquid nitrogen to snap freeze tissue and blend tissue to powder.

2. Add 10 ml genomic DNA extraction buffer in 50 ml tube and put tissue powder in.

3. Invert tube to submerge tissue powder and incubate at 37 deg C. for 1 hour.

4. Add 50 ul proteinase K (20 mg/ml stock), mix gently.

5. Incubate in 50c water bath for 3 hours, shake gently.

6. Let stand in room temperature for 30 min to equilibrate to room temperature.

7. Add 10 ml Phenol, mix gently for 10 min.

8. Centrifuge at 3000 rpm×15 min.

9. Transfer the viscous aqueous phase to a new tube using a wide-pore glass pipette.

10. Repeat phenol extraction for 2 times or more.

11. Add 2 ml ammonium acetate (10M), mix gently.

12. Add a volume of ethanol (in room temperature). Swirl gently and you will see genomic DNA start to form the white mass. Transfer genomic DNA to a new tube by using a “U” shape pipette.

13. Air dry for 5-10 min to drive off ethanol and dissolve in ddH₂O or TE buffer.

The isolated DNA is subject to gel electrophoresis analysis. FIG. 9 is picture of a gel containing the genomic DNA (2^{nd} and 3^{rd} lanes) isolated from a sample of mouse liver.

### EXAMPLE 3

### Freeze-Drying Isolated DNA

The DNA isolated by the processes of Example 1 or 2 is put into a freeze-dryer for about 6 to 8 hours to obtain a DNA powder.

### EXAMPLE 4

### Production of a Monumental Adornment Carrying Isolated DNA

Method 1

In a centrifuge tube, a drop of paraffinic wax having a melting point of 56-58 ° C. is added to the DNA powder obtained from the freeze-drying. After the surface of the wax is solidified, the centrifuge tube is incubated in cold water to speed up the solidification. After about 10 minute, a wax block embedded with the DNA powder is immersed in a phenol-formaldehyde resin. After the resin is solidified, the treated wax block is used in the next step.

Phenol-formaldehyde resin are composed of Fluid I and II, which are purchased from Shanghai Second Medical University Biological Materials Ltd, Zhangjiang. Fluid I contains 40% formaldehyde solution. Fluid II contains 45% resorcinol, 10% sodium hydroxide, and 45% distilled water, among which sodium hydroxide is a catalyst. Immediately before use, Fluid I and II are mixed at 6:7 (drop) for the preparation of Phenol-formaldehyde resin.

The phenol-formaldehyde resin treated wax block is placed in a compartment of an adornment using a pair of tweezers. The wax block can be placed in the compartment directly, such as that illustrated in FIGS. 4 and 5, or indirectly on a carrier and via a support and lock mechanism as that illustrated in FIGS. 6 and 7.

Method 2

Using a biological sample dispensing system, such as Bio-Dot AD1500 (U.S.A.), the isolated DNA sample is directly applied to the exterior surface of the adornment (see FIG. 1) or interior surface of the adornment (FIG. 4). The DNA sample can also be first applied to a carrier, and the carrier is then fixed to the exterior surface of the adornment (FIG. 3) or interior surface of the adornment (FIG. 6) via a fixation mechanism.

An adornment comprising other isolated genetic materials, such as isolated RNA or prion, can also be made using method similar to that illustrated above.

As will be understood by those familiar with the art, the invention may be embodied in other specific forms without departing from the spirit or essential characteristics thereof Likewise, the particular naming and division of the members, features, attributes, and other aspects are not mandatory or significant, and the mechanisms that implement the invention or its features may have different names, divisions and/or formats. Accordingly, the disclosure of the present invention is intended to be illustrative, but not limiting of the scope of the invention, which is set forth in the following claims.

I/we claim:

1. A monumental adornment comprising an isolated genetic material of an organism.

2. The monumental adornment, wherein the isolated genetic material is linked to, coated on or embedded within the monumental adornment.

3. The monumental adornment of claim 1, wherein the isolated genetic material is placed in a compartment of the monumental adornment.

4. The monumental adornment of claim 1, wherein the genetic material is isolated from a human being.

5. The monumental adornment of claim 1, wherein the genetic material is isolated from a non-human animal.

6. The monumental adornment of claim 5, wherein the non-human animal is selected from the group consisting of Mammalia, Ayes, Reptilia, Amphibia, Gnathostomata, Agnatha, Cephalochorda, Thaliacea, Ascidiacea, Appendiculariae, Graptolite, Pterobranchia, Enteropneusta, Asteroidea, Ophiuroidea, Echinoidea, Holothuroidea, Blastoidea, Crinoidea, Archisagittoidea, Sagittoidea, Hyolitha, Tentaculita, Rostroconchia, Scaphopoda, Cephalopoda, Bivalvia, Gastropoda, Aplacophora, Monoplacophora, Polyplacophora, Sipunculidea, Phascolosomatidea, Hirudinea, Oligochaeta, Polychaeta, Paleonemertea, Anopla, Monogonta, Digononta, Gymnolaemata, Phylactolaemata, Articulata, Inarticulata, Phasmida, Aphasmida, Eutardigrada, Mesotardigrada, Heterotardigrada, Malacostraca, Brabchiura, Mystacocarida, Copepoda, Ostracoda, Cirripedia, Branchiopoda, Cephalocarida, Protura, Diplura, Collembola, Insecta, Symphyla, Pauropoda, Diplopoda, Chilopoda, Pycnogonida, Merostomata, Arachnida, Trilobita, Cestoda, Trematoda, Turbellaria, Anthozoa, Tentaculata, Nuda, Scyphozoa, Hydrozoa, Demospongiae, Hexactinellida and Calcarer.

7. The monumental adornment of claim 5, wherein the non-human animal is selected from the group of consisting of a monkey, dog, cat, rabbit, rat, mouse, squirrel, fish, snake, lizard, gecko, bird, pig, tortoise, horse, sheep, insect, procyon lotor and koala.

8. The monumental adornment of claim 1 wherein the isolated genetic material is an isolated nucleic acid molecule or an isolated prion.

9. The monumental adornment of claim 8, wherein the isolated genetic material is an isolated DNA molecule.

10. The monumental adornment of claim 1, wherein the isolated genetic material is processed to increase its stability or freeze-dried prior to being placed in the monumental adornment.

11. The monumental adornment of claim 1 being a piece selected from the group consisting of jewelries, decorative artifacts, and other arts and crafts.

12. The monumental adornment of claim 1, comprising one or more materials selected from the group consisting of gem, agate, amber, crystal, metal, pearl, wood, bone, shell, leather, ceramics, glass, resin, rubber, paper, plastics, textiles and fiber.

13. A method of preparing a monumental adornment, comprising:
a. isolating a genetic material from an organism; and
b. combining the isolated genetic material with at least one material to produce the monumental adornment.

14. The method of claim 13, further comprising processing the isolated genetic material to increase its stability or freeze-drying the isolated genetic material, prior to the combining step.

15. The method of claim 13, wherein the isolated genetic material is linked to, coated on or embedded within the monumental adornment.

16. The method of claim 13, wherein the isolated genetic material is placed in a compartment of the monumental adornment.

17. The method of claim 13, further comprising associating the isolated genetic material with a nano-particle, and combining the nano-particle associated with the isolated genetic material with the at least one material to produce the monumental adornment.

18. The method of claim 13, further comprising associating the isolated genetic material with a carrier, and combining the carrier associated with the isolated genetic material with the at least one material to produce the monumental adornment.

19. The method of claim 13, wherein the isolated genetic material is an isolated DNA molecule.

20. The method of claim 13, wherein the genetic material is isolated from a human being.
